(19) United States
(12) Patent Application Publication
(10) Pub. No.: US 2026/0098346 A1
(43) Pub. Date: Apr. 9, 2026

(54) COMPOSITION, METHOD OF TREATING METAL-CONTAINING LAYER BY USING THE SAME, AND METHOD OF MANUFACTURING SEMICONDUCTOR DEVICE BY USING THE SAME

(71) Applicant: Samsung Electronics Co., Ltd., Suwon-si (KR)

(72) Inventors: Wonsik YOON, Suwon-si (KR); Giho KO, Suwon-si (KR); Sungmin KIM, Suwon-si (KR); Youngchan KIM, Suwon-si (KR); Jina KIM, Suwon-si (KR); Jinhye BAE, Suwon-si (KR); Minhyung CHO, Suwon-si (KR); Cheol HAM, Suwon-si (KR); Kyuyoung HWANG, Suwon-si (KR)

(73) Assignee: Samsung Electronics Co., Ltd., Suwon-si (KR)

(21) Appl. No.: 19/338,741

(22) Filed: Sep. 24, 2025

(30) Foreign Application Priority Data
Oct. 7, 2024 (KR) 10-2024-0135964

(51) Int. Cl. C23G 1/20; C07C 233/47; C23F 1/34; C23F 1/36; C23F 1/38; C23F 1/40; C23G 1/00; C23G 1/18; H01L 21/3213

(52) U.S. Cl. CPC C23G 1/205; C07C 233/47; C23F 1/34; C23F 1/36; C23F 1/38; C23F 1/40; C23G 1/18; H10P 50/667; C23G 1/00

(57) ABSTRACT

Provided are a composition, a method of treating a metal-containing layer by using the same, and a method of manufacturing a semiconductor device by using the same, the composition including an oxidizing agent, an ammonium-based buffer, and an etching controller, wherein the etching controller includes a compound represented by Formula 1. A description of Formula 1 is provided in the specification.

### CROSS-REFERENCE TO RELATED APPLICATION

This application is based on and claims priority under 35 USC § 119 to Korean Patent Application No. 10-2024-0135964, filed on Oct. 7, 2024, in the Korean Intellectual Property Office, the disclosure of which is incorporated by reference herein in its entirety.

### BACKGROUND

### 1. Field

The disclosure relates to a composition, a method of treating a metal-containing layer by using the same, and a method of manufacturing a semiconductor device by using the same.

### 2. Description of the Related Art

To meet consumer demands for excellent performance and affordable prices, an increase in semiconductor device integration and an improvement in reliability are being explored. As the degree of integration of semiconductor devices increases, damage to components of semiconductor devices during the manufacturing process has a greater influence on reliability and electrical characteristics of semiconductor memory devices. In particular, during the manufacturing process of semiconductor devices, various treatment processes, for example, etching and cleaning processes, may be performed on a preset layer (for example, a metal-containing layer), and a need for a composition having an appropriate etching rate and excellent cleaning capability continues to be a benefit for performing effective metal-containing layer treatment processes.

### SUMMARY

Provided are a composition having comparatively excellent etching selectivity and cleaning performance, a method of treating a metal-containing layer by using the same, and a method of manufacturing a semiconductor device by using the same.

Additional aspects will be set forth in part in the description which follows and, in part, will be apparent from the description, or may be learned by practice of the presented embodiments of the disclosure.

According to an aspect of the disclosure, a composition includes:
an oxidizing agent at about 16 wt % to about 50 wt % per 100 wt % of the composition, an ammonium-based buffer, and an etching controller,
the etching controller including a compound represented by Formula 1:

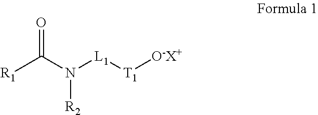

wherein, in Formula 1,
R₁ may be a C₁₂-C₅₀ alkyl group or a C₁₂-C₅₀ alkenyl group,
R₂ may be hydrogen, a C₁-C₅₀ alkyl group, or a C₂-C₅₀ alkenyl group,
L₁ may be a C₁-C₂₀ alkylene group,
T₁ may be a linker represented by one of Formulae 2(1) to 2(3), and
X may be hydrogen, an alkali metal, or an ammonium group,
wherein at least one of methylene groups included in R₁ and R₂ may optionally be substituted with O or S, and
wherein at least one of the hydrogens included in R₁, R₂ and L₁ may optionally be substituted with a halogen atom, a C₁-C₃₀ alkoxy group, or a C₁-C₃₀ alkylthio group, and

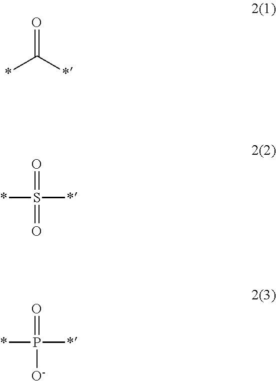

wherein, in Formulae 2(1) to 2(3),
* indicates a binding site to L₁, and
*′ indicates a binding site to O⁻.

According to another aspect of the disclosure, a method of treating a metal-containing layer includes
preparing a substrate, the substrate including the metal-containing layer; and
contacting the metal-containing layer with the composition.

The metal included in the metal-containing layer may include titanium (Ti), indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), or a combination thereof.

The contacting the metal-containing layer with the composition may include at least a portion of the metal-containing layer being etched and cleaned. For example, through the contacting of the metal-containing layer with the composition, at least a portion of the metal-containing layer may be etched and/or cleaned.

The metal-containing layer may include a first region and a second region, and a second etching rate at which the composition etches the second region may be greater than a first etching rate at which the composition etches the first region.

The first region may include at least one of cobalt and copper, and the second region may include titanium nitride.

The contacting of the metal-containing layer with the composition may include residues on the surface of the metal-containing layer being removed so that at least a portion of the metal-containing layer may be cleaned, and the residues may include an etching gas residue, a polymer residue, a metal-containing residue, or a combination thereof.

According to another aspect of the disclosure, a method of manufacturing a semiconductor device includes
preparing a substrate, the substrate including a metal-containing layer,
contacting the metal-containing layer with the composition, and
performing a subsequent manufacturing process.

### BRIEF DESCRIPTION OF THE DRAWING

The above and other aspects, features, and advantages of certain embodiments of the disclosure will be more apparent from the following description taken in conjunction with the accompanying drawings, in which:

FIG. 1 is a diagram illustrating including an etching controller of at least one embodiment bonded to a metal-containing layer;

FIG. 2 is a process flow diagram of an embodiment of a method of manufacturing a semiconductor device;

FIGS. 3 and 4 are drawings briefly illustrating an embodiment of a metal-containing layer treatment method; and

FIGS. 5A to 5J are cross-sectional views illustrating an embodiment of a trench and via hole pattern formation process for forming a bit line electrode.

### DETAILED DESCRIPTION

Reference will now be made in detail to embodiments, examples of which are illustrated in the accompanying drawings, wherein like reference numerals refer to like elements throughout the specification. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. Additionally, when the terms “about” or “substantially” are used in this specification in connection with a numerical value and/or geometric terms, it is intended that the associated numerical value includes a manufacturing tolerance (e.g., ±10%) around the stated numerical value. Further, regardless of whether numerical values and/or geometric terms are modified as “about” or “substantially,” it will be understood that these values should be construed as including a manufacturing or operational tolerance (e.g., ±10%) around the stated numerical values and/or geometry. Further, when referring to as “within a range of” “C to D”, this means C inclusive to D inclusive unless otherwise specified.

### Metal-Containing Layer

A metal-containing layer may include a metal, a metal nitride, a metal oxide, a metal oxynitride, and/or a combination thereof. A metal (or, an elemental metal) included in a metal-containing layer may be an alkali metal (for example, sodium (Na), potassium (K), rubidium (Rb), cesium (Cs), and/or the like), an alkaline earth metal (for example, beryllium (Be), magnesium (Mg), calcium (Ca), strontium (Sr), barium (Ba), and/or the like), a lanthanide metal (for example, lanthanum (La), europium (Eu), terbium (Tb), ytterbium (Yb), and/or the like), a transition metal (for example, scandium (Sc), yttrium (Y), titanium (Ti), zirconium (Zr), hafnium (Hf), vanadium (V), niobium (Nb), tantalum (Ta), chromium (Cr), molybdenum (Mo), tungsten (W), manganese (Mn), iron (Fe), ruthenium (Ru), osmium (Os), cobalt (Co), rhodium (Rh), nickel (Ni), copper (Cu), silver (Ag), zinc (Zn), and/or the like), a post-transition metal (for example aluminum (Al), gallium (Ga), indium (In), thallium (Tl), tin (Sn), bismuth (Bi), and/or the like), and/or a combination thereof.

According to at least one embodiment, the metal included in the metal-containing layer may include titanium (Ti), indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), and/or a combination thereof.

According to some embodiments, the metal-containing layer may include two or more different types of metals.

For example, according to some embodiments, the metal-containing layer may include:
i) titanium (Ti), and
ii) indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), and/or a combination thereof.

For example, the metal-containing layer may include aluminum (Al), titanium (Ti), lanthanum (La), cobalt (Co), copper (Cu), or a combination thereof.

According to at least one embodiment, the metal-containing layer may include titanium.

According to some embodiments, the metal-containing layer may include cobalt.

According to some embodiments, the metal-containing layer may include copper.

According to some embodiments, the metal-containing layer may include titanium and cobalt.

According to some embodiments, the metal-containing layer may include titanium and copper.

For example, in at least one embodiment, the metal-containing layer may include a metal, a metal nitride, a metal oxide, a metal oxynitride, or a combination thereof, and each of the metal, the metal of the metal nitride, the metal of the metal oxide, and the metal of the metal oxynitride may include titanium (Ti), indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), or a combination thereof.

According to some embodiments, the metal-containing layer may include a metal nitride as described above.

According to some embodiments, the metal-containing layer may include a metal as described above (e.g., at least one of cobalt and copper).

According to some embodiments, the metal-containing layer may include a metal nitride and a metal (e.g., at least one of cobalt and copper). For example, the metal included in the metal nitride and the metal may be different from each other.

According to some embodiments, the metal-containing layer may include a metal nitride and a metal (e.g., at least one of cobalt and copper), and the metal included in the metal nitride may include indium, titanium, aluminum, lanthanum, scandium, gallium, zinc, hafnium, and/or a combination thereof.

In some embodiments, the metal-containing layer may include titanium nitride and a metal (e.g., at least one of cobalt and copper), wherein the titanium nitride may optionally further include indium, aluminum, lanthanum, scandium, gallium, hafnium, zinc, tungsten, silicon, and/or a combination thereof.

According to some embodiments, the metal-containing layer may include at least one of titanium nitride (TiN), titanium nitride further including aluminum (for example, titanium aluminum nitride or TiAlN), titanium nitride further including lanthanum, and/or the like.

In some embodiments, the metal-containing layer may include a metal oxide. The metal included in the metal oxide may include titanium, aluminum, lanthanum, scandium, gallium, hafnium, and/or a combination thereof. For example, the metal-containing layer may include aluminum oxide (for example, Al₂O₃), indium gallium zinc oxide (IGZO), and/or the like.

In some embodiments, the metal-containing layer may include the metal nitride and the metal oxide.

In some embodiments, the metal-containing layer may further include, in addition to the metal, a metalloid (for example, boron (B), silicon (Si), germanium (Ge), arsenic (As), antimony (Sb), tellurium (Te), and/or the like), a non-metal (for example, nitrogen (N), phosphorus (P), oxygen (O), sulfur(S), selenium (Se), and/or the like), and/or a combination thereof.

For example, the metal-containing layer may further include silicon oxide.

According to at least one embodiment, the metal-containing layer may include:
a) i) titanium nitride or ii) titanium nitride further including indium, aluminum, lanthanum, scandium, gallium, hafnium, zinc, tungsten, silicon, and/or a combination thereof, and
b) at least one of cobalt and copper.

The metal-containing layer may be a single-layer structure including one or more materials, or a multi-layer structure including different materials. The plurality of layers included in the multi-layer structure may be vertically stacked or horizontally arranged. The single-layer structure and multi-layer structure may have various three-dimensional patterns (e.g., via holes, trenches, and/or the like).

According to at least one embodiment, the metal-containing layer may include a first region with a first etching rate and a second region with a second etching rate; the second etching rate may be greater than the first etching rate for the composition. During a treatment process (e.g., etching, cleaning process, and/or the like) for the metal-containing layer, at least a portion of the first region and at least a portion of the second region may each come into contact with the composition, and because the second etching rate is greater than the first etching rate, the second region may be etched faster than the first region. In at least one embodiment, the first etching rate may be 0 such that the first region may not be etched.

For example, the first region may include a metal, a metal oxide (e.g., aluminum oxide), silicon oxide, and/or a combination thereof.

According to at least one embodiment, the first region may include at least one of cobalt and copper.

In some embodiments, the second region may include a metal nitride (e.g., titanium nitride).

In some embodiments, the second region may include i) titanium nitride (TiN), ii) titanium nitride further including indium, aluminum, lanthanum, scandium, gallium, zinc, hafnium, or any combination thereof (e.g., TiAlN), and/or iii) a combination thereof.

In some embodiments, each of the first region and the second region may include i) titanium nitride, ii) titanium nitride further including indium, aluminum, lanthanum, scandium, gallium, zinc, hafnium, and/or a combination thereof, and/or iii) a combination thereof.

In some embodiments, the first region may include at least one of cobalt and copper, and the second region may not include cobalt and copper.

In some embodiments, the first region may include at least one of cobalt and copper, and the second region may include i) titanium nitride (TiN), ii) titanium nitride further including indium, aluminum, lanthanum, scandium, gallium, zinc, hafnium, and/or a combination thereof (e.g., TiAlN), and/or iii) a combination thereof.

In some embodiments, the first region may include at least one of cobalt and copper, and the second region may include titanium nitride (TiN), titanium nitride further including aluminum (TiAlN), and/or a combination thereof.

In some embodiments, the first region may include at least one of a cobalt layer and a copper layer, and the second region may include a titanium nitride layer (TiN layer), a titanium nitride layer further including aluminum (e.g., a titanium aluminum nitride layer or a TiAlN layer), and/or a combination thereof.

In some embodiments, the first region may be a cobalt layer, a copper layer, and/or a combination thereof, and the second region may be a titanium nitride layer (TIN layer) or a titanium nitride layer further including aluminum (e.g., a titanium aluminum nitride layer or a TiAlN layer).

Etching of a layer herein may refer to the removal of at least a portion of a material constituting the layer.

### Composition

The composition may include an oxidizing agent, an ammonium-based buffer, and an etching controller.

The composition may be used in various treatment processes for the metal-containing layer described herein, for example, an etching process, a cleaning process, and/or the like.

The composition may further include a solvent, such as water.

According to at least one embodiment, the composition may not include an abrasive.

According to some embodiments, the composition may not include fluorine (F).

### Oxidizing Agent

The oxidizing agent is configured to etch at least a portion of the metal-containing layer by oxidizing at least a portion of metal in the metal-containing layer to form a soluble complex. For example, in at least one embodiment, the complex may be a water-soluble complex. In at least one embodiment, the oxidizing agent may include at least one of hydrogen peroxide, nitric acid, ammonium persulfate, and/or a combination thereof.

In at least one embodiment, the oxidizing agent may include hydrogen peroxide. In some embodiments, the oxidizing agent may be hydrogen peroxide.

The amount (weight) of the oxidizing agent may be, for example, per 100 wt % of the composition, about 16 wt % to about 50 wt %, about 18 wt % to about 50 wt %, about 20 wt % to about 50 wt %, about 22 wt % to about 50 wt %, about 25 wt % to about 50 wt %, about 16 wt % to about 45 wt %, about 18 wt % to about 45 wt %, about 20 wt % to about 45 wt %, about 22 wt % to about 45 wt %, about 25 wt % to about 45 wt %, about 16 wt % to about 40 wt %, about 18 wt % to about 40 wt %, about 20 wt % to about 40 wt %, about 22 wt % to about 40 wt %, about 25 wt % to about 40 wt %, about 16 wt % to about 35 wt %, about 18 wt % to about 35 wt %, about 20 wt % to about 35 wt %, about 22 wt % to about 35 wt %, about 25 wt % to about 35 wt %, about 16 wt % to about 30 wt %, about 18 wt % to about 30 wt %, about 20 wt % to about 30 wt %, about 22 wt % to about 30 wt %, about 25 wt % to about 30 wt %, about 16 wt % to about 27 wt %, about 18 wt % to about 27 wt %, about 20 wt % to about 27 wt %, about 22 wt % to about 27 wt %, and/or about 25 wt % to about 27 wt %.

When the amount range of the oxidizing agent satisfies these ranges described above, the composition may have excellent etching selectivity and excellent cleaning performance at the same time.

### Ammonium-Based Buffer

The ammonium-based buffer is configured to maintain a high concentration of anions from the oxidizing agent and to stabilize a water-soluble complex generated by oxidizing at least a portion of metal in the metal-containing layer by the anions. By using such an ammonium-based buffer, at least a portion of the metal-containing layer can be more effectively etched.

The ammonium-based buffer may include an ammonium group.

According to at least one embodiment, the ammonium-based buffer may include an ammonium group represented by N(A₁₁)(A₁₂)(A₁₃)(A₁₄), wherein A₁₁ to A₁₄ may each independently be hydrogen, a C₁-C₃₀ alkyl group, a C₂-C₃₀ alkenyl group, a C₃-C₃₀ carbocyclic group, and/or a C₁-C₃₀ heterocyclic group.

For example, A₁₁ to A₁₄ may each independently be hydrogen or a C₁-C₁₀ alkyl group.

In some embodiments, the ammonium-based buffer may not include fluorine (F). In case that the ammonium-based buffer does not include fluorine, acceleration of corrosion at the surface of the metal-containing layer may be substantially reduced and/or prevented, and the metal-containing layer treatment process using the composition may be performed in a safe and environmentally friendly atmosphere.

In some embodiments, the ammonium-based buffer may include hydroxide, acetate, bicarbonate, benzoate, carbonate, formate, nitrate, hydrogensulfate, carbamate, sulfamate, citrate, phosphate, sulfite, sulfobenzoate, oxalate, lactate, tartrate, dihydrogencitrate, glutamate, salicylate, bioxalate, octanoate, propionate, glycolate, gluconate, and/or the like. The term “phosphate” used herein may include tribasic phosphate, monohydrogenphosphate, dihydrogenphosphate, or any combination thereof.

In some embodiments, the ammonium-based buffer may include phosphate or hydroxide.

In some embodiments, the ammonium-based buffer may include a compound represented by Formula 11-1, a compound represented by Formula 11-2, a compound represented by Formula 11-3, a compound represented by Formula 11-4, and/or a combination thereof:

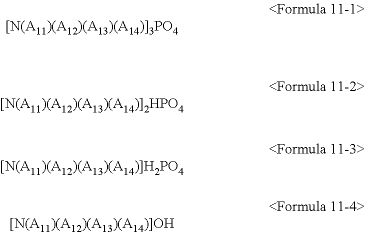

Each of A₁₁ to A₁₄ among Formulae 11-1 to 11-4 is as described in connection with A₁₁ to A₁₄ in the ammonium group represented by N(A₁₁)(A₁₂)(A₁₃)(A₁₄) in this specification.

In some embodiments, the ammonium-based buffer may include at least one of tribasic ammonium phosphate ((NH₄)₃PO₄), diammonium monohydrogen phosphate ((NH₄)₂HPO₄), ammonium dihydrogen phosphate ((NH₄)H₂PO₄), tris(tetramethylammonium) phosphate ([N(CH₃)₄]₃PO₄), bis(tetramethylammonium) monohydrogen phosphate ([N(CH₃)₄]₂HPO₄), tetramethylammonium dihydrogen phosphate ([N(CH₃)₄]H₂PO₄), ammonium hydroxide, and/or a combination thereof.

The amount (weight) of the ammonium-based buffer may be, for example, per 100 wt % of the composition, about 0.01 wt % to about 10 wt %, about 0.05 wt % to about 10 wt %, about 0.1 wt % to about 10 wt %, about 0.3 wt % to about 10 wt %, about 0.5 wt % to about 10 wt %, about 0.01 wt % to about 7 wt %, about 0.05 wt % to about 7 wt %, about 0.1 wt % to about 7 wt %, about 0.3 wt % to about 7 wt %, about 0.5 wt % to about 7 wt %, about 0.01 wt % to about 4 wt %, about 0.05 wt % to about 4 wt %, about 0.1 wt % to about 4 wt %, about 0.3 wt % to about 4 wt %, about 0.5 wt % to about 4 wt %, about 0.01 wt % to about 2 wt %, about 0.05 wt % to about 2 wt %, about 0.1 wt % to about 2 wt %, about 0.3 wt % to about 2 wt %, about 0.5 wt % to about 2 wt %, about 0.01 wt % to about 1 wt %, about 0.05 wt % to about 1 wt %, about 0.1 wt % to about 1 wt %, about 0.3 wt % to about 1 wt %, about 0.5 wt % to about 1 wt %, about 0.01 wt % to about 0.7 wt %, about 0.05 wt % to about 0.7 wt %, about 0.1 wt % to about 0.7 wt %, about 0.3 wt % to about 0.7 wt %, about 0.5 wt % to about 0.7 wt %, about 0.01 wt % to about 0.5 wt %, about 0.05 wt % to about 0.5 wt %, about 0.1 wt % to about 0.5 wt %, and/or about 0.3 wt % to about 0.5 wt %.

When the amount range of the ammonium-based buffer satisfies the ranges described above, the composition may have excellent etching selectivity and excellent cleaning performance at the same time.

### Etching Controller

The etching controller may be configured to interact with various metal atoms in the metal-containing layer, which is the target layer, to control the etching rate. In addition, the etching controller may remove residues generated during a metal-containing layer formation process and/or a patterning process.

The above etching controller may include the compound represented by Formula 1:

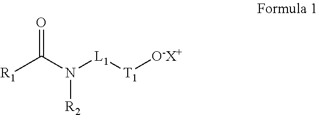

wherein, in Formula 1,
R₁ may be a C₁₂-C₅₀ alkyl group or a C₁₂-C₅₀ alkenyl group,
R₂ may be hydrogen, a C₁-C₅₀ alkyl group, or a C₂-C₅₀ alkenyl group,
L₁ may be a C₁-C₂₀ alkylene group, and
T₁ may be a linker represented by one of Formulae 2(1) to 2(3),
X may be hydrogen, an alkali metal, or an ammonium group,
at least one of the methylene groups included in R₁ and R₂ may optionally be substituted with oxygen (O) or sulfur(S), and
at least one of the hydrogens included in R₁, R₂ and L₁ may optionally be substituted with a halogen atom, a C₁-C₃₀ alkoxy group, or a C₁-C₃₀ alkylthio group,

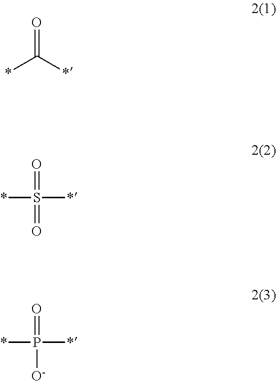

in the above Formulae 2(1) to 2(3),
* indicates a binding site to L₁, and
*′ indicates a binding site to O⁻ in Formula 1. In Formula 2(3), O⁻ may bind to a monovalent cation included in the composition, for example H⁺.

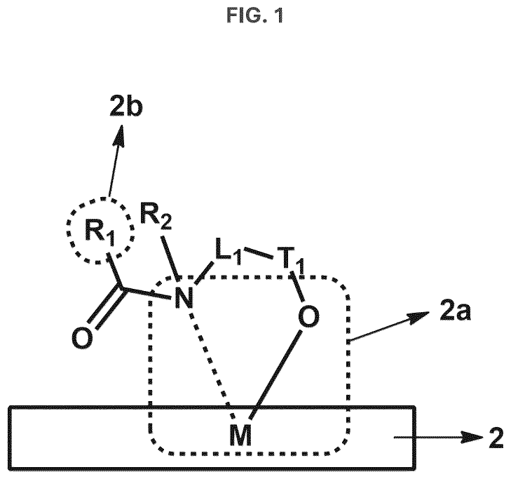

FIG. 1 is a diagram illustrating including an etching controller of at least one embodiment bonded to a metal-containing layer. As shown in FIG. 1, the etching controller may be configured to form a metal complex with a metal included in the metal-containing layer 2. For example, O⁻ and the nitrogen (N) of amide in Formula 1 may form a strong bond (see “2a” of FIG. 1) with the metal M (e.g., cobalt, copper, and/or the like) included in the metal-containing layer 2, and thereby the compound represented by Formula 1 may be effectively fixed on the surface of the metal-containing layer 2. In addition, because R₁ (see “2b” of FIG. 1) defined as described above is a hydrophobic group having a relatively long chain, the compound represented by Formula 1 may provide a hydrophobic protective layer on the surface of the metal-containing layer 2. Therefore, by using a composition including a compound represented by Formula 1, the etching rate may be selectively controlled depending on the metal of the metal-containing layer, and at the same time, residues generated during the metal-containing layer formation process and/or patterning process may be effectively removed.

According to at least one embodiment, R₁ in Formula 1 may be:
1) a C₁₂-C₃₀ alkyl group or a C₁₂-C₃₀ alkenyl group, or
2) a C₁₂-C₂₀ alkyl group or a C₁₂-C₂₀ alkenyl group, or
3) a C₁₂-C₁₇ alkyl group or a C₁₂-C₁₇ alkenyl group, or
4) a C₁₃-C₅₀ alkyl group or a C₁₃-C₅₀ alkenyl group, or
5) a C₁₃-C₃₀ alkyl group or a C₁₃-C₃₀ alkenyl group, or
6) a C₁₃-C₂₀ alkyl group or a C₁₃-C₂₀ alkenyl group, or
7) a C₁₃-C₁₇ alkyl group or a C₁₃-C₁₇ alkenyl group,
8) a C₁₅-C₅₀ alkyl group or a C₁₅-C₅₀ alkenyl group, or
9) a C₁₅-C₃₀ alkyl group or a C₁₅-C₃₀ alkenyl group, or
10) a C₁₅-C₂₀ alkyl group or a C₁₅-C₂₀ alkenyl group, or
11) a C₁₅-C₁₇ alkyl group or a C₁₅-C₁₇ alkenyl group.

In some embodiments, R₂ in Formula 1 may be:
i) hydrogen, a C₁-C₂₀ alkyl group, or a C₂-C₂₀ alkenyl group, or
ii) hydrogen, a C₁-C₁₀ alkyl group, or a C₂-C₁₀ alkenyl group, or
iii) hydrogen, a C₁-C₅ alkyl group, or a C₂-C₅ alkenyl group, or
iv) a hydrogen or a methyl group.

In some embodiments, L₁ in Formula 1 may be:
i) a C₁-C₁₀ alkylene group,
ii) a C₁-C₄ alkylene group,
iii) a C₁-C₂ alkylene group, or
iv) a C₁ alkylene group (a methylene group).

In some embodiments, T₁ in Formula 1 may be a linker represented by Formula 2(1).

According to some embodiments, in Formula 1, X may be hydrogen, Na, K, or N (A₁) (A₂) (A₃) (A₄), and A₁ to A₄ may each independently be hydrogen, a C₁-C₃₀ alkyl group, a C₂-C₃₀ alkenyl group, a C₃-C₃₀ carbocyclic group, or a C₁-C₃₀ heterocyclic group. For example, A₁ to A₄ may each independently be hydrogen or a C₁-C₁₀ alkyl group.

In some embodiments, at least one of the methylene groups (e.g., one or two methylene groups (CH₂) included in R₁ and R₂ of Formula 1 may optionally be substituted with O or S.

In some embodiments, at least one of the hydrogens included in R₁, R₂ and L₁ in Formula 1 may optionally be substituted with a halogen atom (e.g., −F, −Cl, −Br, or the like), a C₁-C₃₀ alkoxy group (e.g., a C₁-C₁₀ alkoxy group), or a C₁-C₃₀ alkylthio group (e.g., a C₁-C₁₀ alkylthio group).

In some embodiments, the etching controller may include at least one of Compounds 1 to 4:

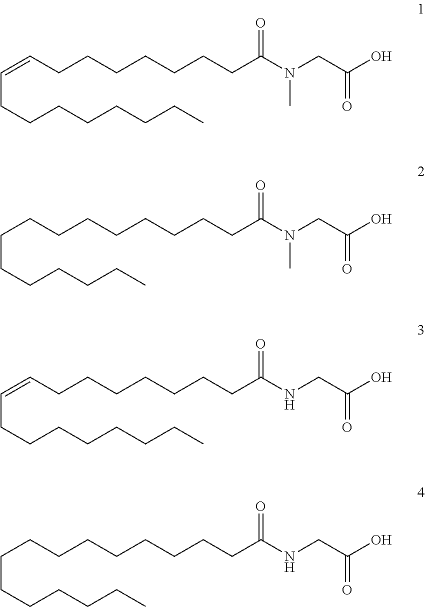 [SMILES: CCCCCCCC/C=C\CCCCCCCC(=O)N(C)CC(=O)O ; CCCCCCCCCCCCCCCC(=O)N(C)CC(=O)O ; CCCCCCCC/C=C\CCCCCCCC(=O)NCC(=O)O ; CCCCCCCCCCCCCCCC(=O)NCC(=O)O]

In some embodiments, the amount of the etching controller may be, per 100 wt % of the composition, about 0.001 wt % to about 10 wt %, about 0.01 wt % to about 10 wt %, about 0.1 wt % to about 10 wt %, about 0.2 wt % to about 10 wt %, about 0.001 wt % to about 5 wt %, about 0.01 wt % to about 5 wt %, about 0.1 wt % to about 5 wt %, about 0.2 wt % to about 5 wt %, about 0.001 wt % to about 1 wt %, about 0.01 wt % to about 1 wt %, about 0.1 wt % to about 1 wt %, about 0.2 wt % to about 1 wt %, about 0.001 wt % to about 0.5 wt %, about 0.01 wt % to about 0.5 wt %, about 0.1 wt % to about 0.5 wt %, about 0.2 wt % to about 0.5 wt %, about 0.001 wt % to about 0.2 wt %, about 0.01 wt % to about 0.2 wt %, and/or about 0.1 wt % to about 0.2 wt %.

The composition as described above may have a pH of about 1.0 to about 10.0, about 3.0 to about 10.0, about 5.0 to about 10.0, about 7.0 to about 10.0, about 3.0 to about 8.0, about 5.0 to about 8.0, or about 7.0 to about 8.0. In case that the composition has such pH ranges as described above, the interaction between the etching controller and the metal atoms in the metal-containing layer as described above may occur more smoothly.

According to at least one embodiment, the composition may be used in a treatment process, for example, an etching process, a cleaning process, and/or the like, for the metal-containing layer as described herein.

Alternatively, the composition may also be used as an etching by-product remover, a post-etch process by-product remover, an ashing process by-product remover, a cleaning composition, a photoresist (PR) remover, an etching composition for packaging process, a cleaner for packaging process, a wafer adhesive material remover, an etchant, a post-etch residue stripper, an ash residue cleaner, a photoresist residue stripper, a post-chemical mechanical polishing (CMP) cleaner, and/or the like.

### Metal-Containing Layer Treatment Method and Semiconductor Device Manufacturing Method

A metal-containing layer may be effectively treated by using the composition described above.

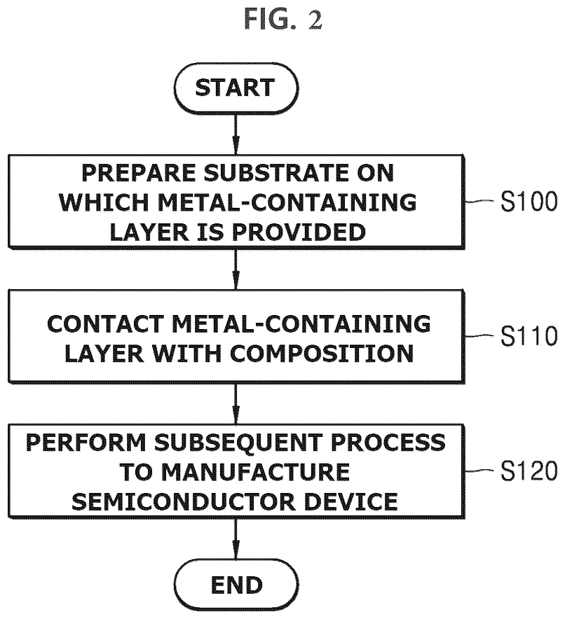

Referring to FIG. 2, an example of the method of treating a metal-containing layer may include: preparing a substrate on which a metal-containing layer is provided (S100); and contacting the metal-containing layer with the composition described herein (S110).

The metal-containing layer is as described herein.

For example, the metal included in the metal-containing layer may include titanium (Ti), indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), and/or a combination thereof.

In some embodiments, the metal-containing layer may include a metal, a metal nitride, a metal oxide, a metal oxynitride, and/or a combination thereof.

In some embodiments, the metal-containing layer may include a metal, a metal nitride, a metal oxide, a metal oxynitride, or any combination thereof, and each of the metal, the metal of the metal nitride, the metal of the metal oxide, and the metal of the metal oxynitride may include titanium (Ti), indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), or any combination thereof.

In some embodiments, the metal-containing layer may include titanium nitride.

In some embodiments, the metal-containing layer may include at least one of cobalt and copper.

According to at least one embodiment, due to the contacting the metal-containing layer with the composition, at least a portion of the metal-containing layer may be etched and cleaned.

Regarding the composition, i) the oxidizing agent oxidizes at least a portion of the metal of the metal-containing layer to form a water-soluble complex, thereby etching at least a portion of the metal-containing layer, ii) the ammonium-based buffer functions to effectively etch at least a portion of the metal-containing layer by maintaining a high concentration of anions from the oxidizing agent and by stabilizing a water-soluble complex generated by oxidizing at least a portion of metal in the metal-containing layer by the anions, and iii) the etching controller including the compound represented by Formula 1, in which O⁻ and nitrogen (N) of amide are enabled to form a strong bond with metal (for example, cobalt, copper, or the like) included in the metal-containing layer and R₁ is a hydrophobic group having a relatively long chain, may selectively control an etching rate according to metal of the metal-containing layer and simultaneously to effectively remove residues generated during a layer formation process and/or a patterning process of the metal-containing layer. Therefore, the composition as described above may be usefully used in various treatment processes for the metal-containing layer.

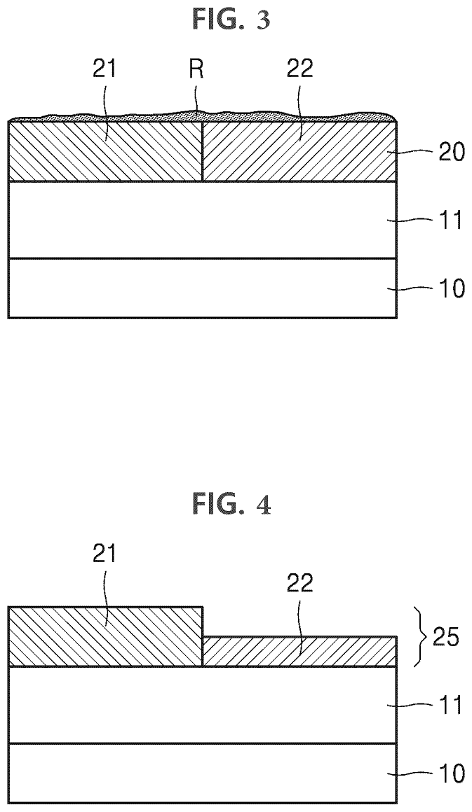

FIGS. 3 and 4 are drawings briefly illustrating at least one embodiment of a metal-containing layer treatment method.

Referring to FIG. 3, provided is a substrate 10 on which a metal-containing layer 20 is provided. An interlayer 11 may be placed between the substrate 10 and the metal-containing layer 20. Although not shown in FIG. 2, circuitry elements (e.g., transistor gates, metal lines, impurity regions, semiconductor layers, etc.) may be arranged within the substrate 10, on the substrate 10, and/or between the substrate 10 and the interlayer 11. According to at least one embodiment, the metal-containing layer 20 may be directly placed on the substrate 10, and the interlayer 11 may be omitted.

The metal-containing layer 20 may include a first region 21 and a second region 22. The first region 21 and the second region 22 may be arranged spaced apart from each other or at least a portion of the first region 21 and at least a portion of the second region 22 may come into contact with each other, and the metal-containing layer 20 may have various three-dimensional patterns. The second etching rate at which the composition etches the second region 22 may be greater than the first etching rate at which the composition etches the first region 21. For example, the first etching rate may be 0, and the first region 21 may not be etched.

Referring to FIG. 4, the composition may be used to etch the metal-containing layer 20 to etch at least a portion of the second region 22, thereby forming a pattern of the metal-containing layer 25. This process may be performed by contacting at least a portion of the first region 21 and at least a portion of the second region 22 with the composition described herein.

The composition may etch only at least a portion of the second region 22 without etching the first region 21. Alternatively, the composition may etch at least a portion of the first region 21 and a greater portion of the second region 22. Referring to FIG. 4, the pattern of the metal-containing layer 25 formed after etching may include at least a portion of the second region 22, and various other modifications can be made, for example, in at least one embodiment, the etching process may be performed such that the second region 22 of the pattern of the metal-containing layer 25 is completely removed.

In some embodiments, the first region 21 may include at least one of cobalt and copper.

In some embodiments, the second region 22 may include a metal nitride (e.g., titanium nitride).

In some embodiments, the second region 22 may include i) titanium nitride (TIN), ii) titanium nitride further including indium, aluminum, lanthanum, scandium, gallium, zinc, hafnium, and/or a combination thereof (e.g., TiAlN), and/or iii) a combination thereof.

In some embodiments, each of the first region 21 and the second region 22 may include i) titanium nitride, ii) titanium nitride further including indium, aluminum, lanthanum, scandium, gallium, zinc, hafnium, and/or any combination thereof, and/or iii) a combination thereof.

In some embodiments, the first region 21 may include at least one of cobalt and copper, and the second region 22 may not include cobalt and copper.

In some embodiments, the first region 21 may include at least one of cobalt and copper, and the second region 22 may include i) titanium nitride (TiN), ii) titanium nitride further including indium, aluminum, lanthanum, scandium, gallium, zinc, hafnium, and/or a combination thereof (e.g., TiAlN), and/or iii) a combination thereof.

In some embodiments, the first region 21 may include at least one of cobalt and copper, and the second region 22 may include titanium nitride (TiN), titanium nitride further including aluminum (TiAlN), and/or a combination thereof.

In some embodiments, the first region 21 may include at least one of a cobalt layer and a copper layer, and the second region 22 may include a titanium nitride layer (TiN layer), a titanium nitride layer further including aluminum (e.g., a titanium aluminum nitride layer or a TiAlN layer), and/or a combination thereof.

In some embodiments, the first region 21 may be a cobalt layer, and the second region 22 may be a titanium nitride layer (TiN layer) or a titanium nitride layer further including aluminum (e.g., a titanium aluminum nitride layer or a TiAlN layer).

In some embodiments, the first region 21 may be a copper layer, and the second region 22 may be a titanium nitride layer (TiN layer) or a titanium nitride layer further including aluminum (e.g., a titanium aluminum nitride layer or a TiAlN layer).

In some embodiments, due to contacting the metal-containing layer 20 with the composition, the residue R on the surface of the metal-containing layer 20 is removed, thereby cleaning at least a portion of the metal-containing layer 20, thereby forming the pattern of the metal-containing layer 25 in which no residue R remains, as in FIG. 4.

The residue R may be a by-product generated during the formation and/or patterning of the metal-containing layer 20 and may remain on the surface of the metal-containing layer 20 and/or the pattern of the metal-containing layer 25 and cause an increase in electrical resistance and/or an electrical short between electrical wirings. The residue R may be an etching residue generated as a result of etching, and may include, for example, an etching gas residue, a polymer residue, a metal-containing residue, or any combination thereof.

The etching gas residue may be a residue derived from an etching gas used for dry etching. The etching gas may be, for example, a fluorocarbon gas. For example, the etching gas may include CHF₃, C₂F₆, CF₄, C₄F₈, C₂HF₅, and/or the like. The etching gas residue may include the etching gas itself and/or a reaction product with any material that came into contact with the etching gas during an etching process using the etching gas.

The polymer residue may be a polymer derived from various organic materials included in a photoresist, dielectric layer, a buffer layer, a diffusion barrier layer, or the like used in the production and/or patterning of the metal-containing layer 20. For example, the polymer residue may be a polymer including carbon, silicon, fluorine, or any combination thereof.

The metal-containing residue may be any residue including metal separated from the metal-containing layer during the production and/or patterning of the metal-containing layer 20.

Referring to FIG. 2, a method of manufacturing a semiconductor device according to at least one embodiment may include preparing a substrate on which a metal-containing layer is provided (S100), contacting the metal-containing layer with the composition (S110), and performing a subsequent manufacturing process to manufacture a semiconductor device (S120). For example, the subsequent manufacturing process may include a deposition process, an etching process, a patterning process, a cleaning process, a dividing process, etc.

For example, the preparing a substrate on which the metal-containing layer is provided (S100) and the contacting the metal-containing layer with the composition (S110) may be used in a trench and via hole pattern formation process for forming a bit line electrode in a method of manufacturing a semiconductor device.

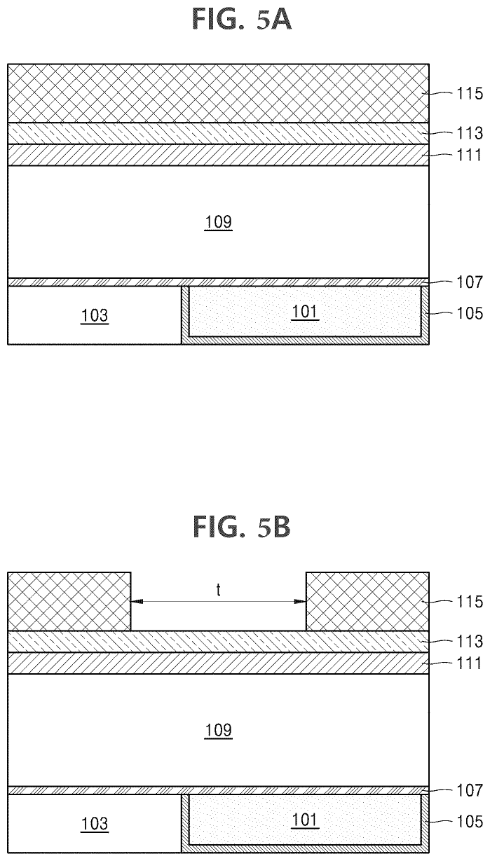

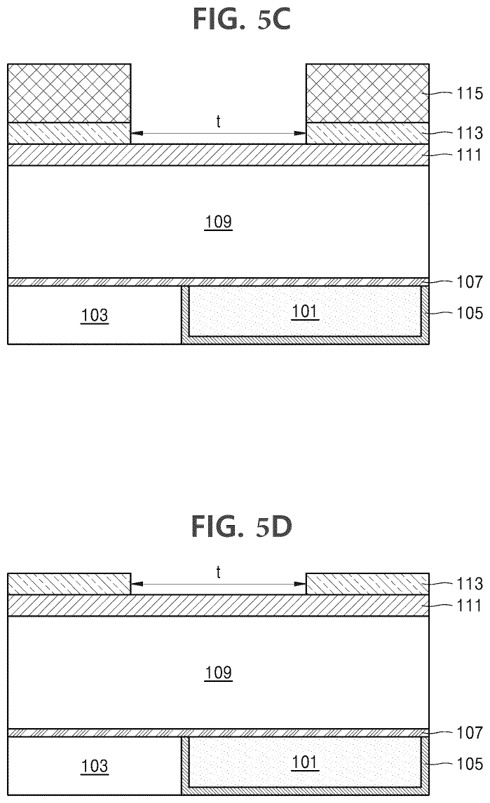

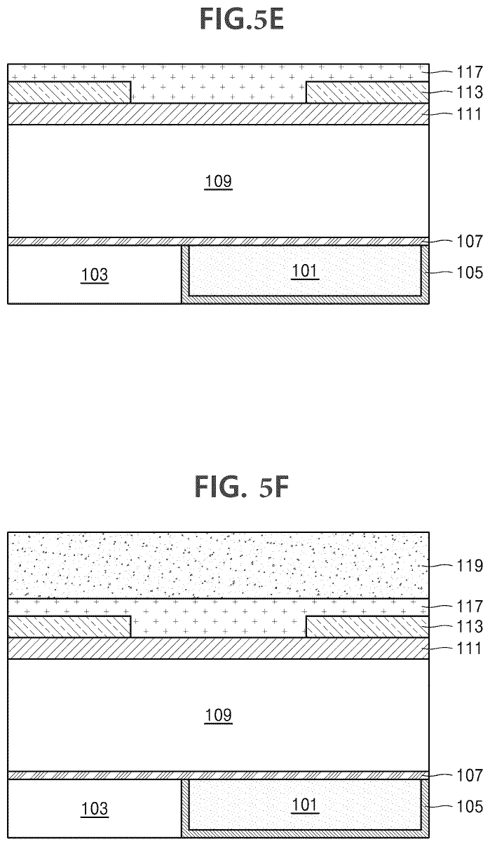

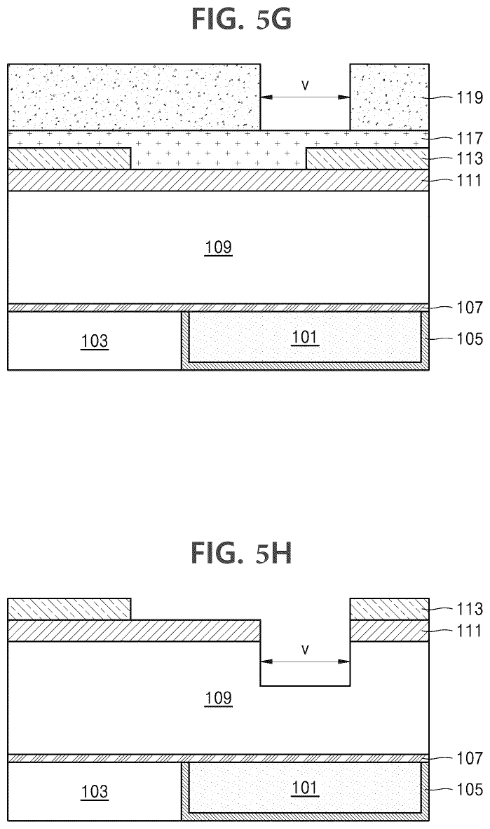

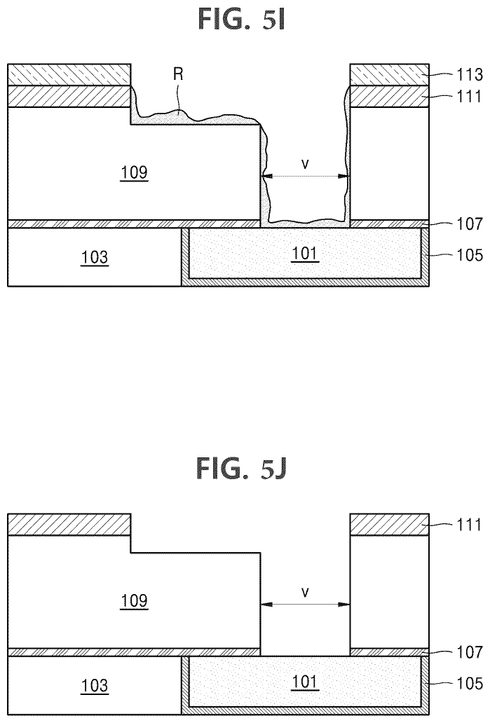

Hereinafter, with reference to FIGS. 5A to 5J, an example of at least one embodiment of a trench and via hole pattern formation process for forming a bit line electrode by using the composition will be described.

FIG. 5A illustrates a portion of a semiconductor substrate (transistors, and the like not shown) including a first dielectric layer 103 and the metal layer 101. The metal layer 101 may include, for example, at least one of copper and cobalt. A first diffusion barrier layer 105 may be placed between the first dielectric layer 103 and the metal layer 101. The first diffusion barrier layer 105 may include, for example, tantalum, titanium, tungsten, tantalum nitride, titanium nitride, tungsten nitride, and/or a combination thereof.

A second diffusion barrier layer 107 may be arranged on the first dielectric layer 103 and the metal layer 101. The second diffusion barrier layer 107 may include, for example, silicon nitride, nitrogen-doped silicon carbide, or aluminum oxide.

A second dielectric layer 109 may be placed on the second diffusion barrier layer 107. The second dielectric layer 109 may include, for example, an ultra-low K(ULK) dielectric or silicon oxide.

A buffer layer 111, which is mechanically robust, may be placed on the second dielectric layer 109 to prevent damage to the second dielectric layer 109 when depositing a hard mask layer 113. The buffer layer 111 may include, for example, tetraethyl orthosilicate (TEOS), carbon-doped silicon oxide (SiCOH), and/or the like.

The hard mask layer 113 may be placed on the buffer layer 111. The hard mask layer 113 may include i) titanium nitride (TiN), ii) titanium nitride further including indium, aluminum, lanthanum, scandium, gallium, zinc, hafnium, and/or a combination thereof (e.g., TiAlN), and/or iii) a combination thereof. For example, the hard mask layer 113 may include TiN.

A first photoresist 115 may be placed on the hard mask layer 113.

Next, the first photoresist 115 is patterned to form a pattern of the first photoresist 115 having a first opening having a width t as illustrated in FIG. 5B, and then the hard mask layer 113 is etched according to the pattern of the first photoresist 115 to open a portion of the buffer layer 111 as illustrated in FIG. 5C, and then, for example, the pattern of the first photoresist 115 is removed using ashing as illustrated in FIG. 5D to form a pattern of the hard mask layer 113 which is exposed.

Next, as shown in FIG. 5E, a filler layer 117 may be formed to cover the pattern of the hard mask layer 113, thereby filling the opening of the pattern of the hard mask layer 113. The filler layer 117 may include, for example, hydrogen silsesquioxane (HSQ) or methyl silsesquioxane (MSQ).

Thereafter, as shown in FIG. 5F, a second photoresist 119 is formed on the filler layer 117, and then the second photoresist 119 is patterned to form a pattern of the second photoresist 119 having a second opening having a width v, as shown in FIG. 5G, and, for example, by using reactive ion etching (RIE), and/or the like, the filler layer 117, a portion of the pattern of the hard mask layer 113, a portion of the buffer layer 111, and a portion of the second dielectric layer 109 located under the pattern of the second photoresist 119, are etched to partially form a via hole, as shown in FIG. 5H, and then the pattern of the second photoresist 119 and the filler layer 117 are removed.

Next, as illustrated in FIG. 5I, according to the pattern of the hard mask layer 113, the buffer layer 111, the second dielectric layer 109, and second diffusion barrier layer 107 are etched by using, for example, a dry etching process until a via hole reaches the metal layer 101, thereby forming a trench and via hole pattern. The etching gas used in the dry etching process may be, for example, a fluorocarbon gas (e.g., CHF₃, C₂F₆, CF₄, C₄F₈, C₂HF₅, and/or the like).

As a result of the dry etching, a large amount of residue R may exist on the inner wall of the trench and via hole pattern, as shown in FIG. 5I. The residue R may include etching gas residue, polymer residue, metal-containing residue, or any combination thereof. The etching gas residue may include the etching gas itself and/or a reaction product with any material (e.g., a material included in the buffer layer 111, the second dielectric layer 109, or the like) that come into contact with the etching gas during an etching process using the etching gas. The polymer residue may be a polymer derived from various organic substances included in the second photoresist 119, the second dielectric layer 109, the buffer layer 111, the second diffusion barrier layer 107, or the like. For example, the polymer residue may be a polymer including carbon, silicon, fluorine, or any combination thereof. The metal-containing residue may be, for example, a residue including a metal included in the pattern of the hard mask layer 113.

The residue R in FIG. 5I may increase the electrical resistance of the semiconductor element and/or may cause an electrical short circuit of the bit line electrode to be formed later, and therefore is removed. Meanwhile, in order to simplify the process, it is beneficial to simultaneously remove the residue R and the pattern of the hard mask layer 113. In addition, the metal layer 101 should not be substantially damaged when the residue R and the pattern of the hard mask layer 113 are removed.

To this end, by bringing a composition including an oxidizing agent, an ammonium-based buffer, and an etching controller as described above into contact with the substrate of FIG. 5I including the metal-containing layer including the pattern of the hard mask layer 113 and the metal layer 101, the substrate of FIG. 5J may be manufactured in which i) the residue R generated on the inner wall of the trench and via hole pattern is removed, ii) the pattern of the hard mask layer 113 is removed, and iii) the metal layer 101 is substantially undamaged. Although not intended to be limited by a specific theory, for example, the pattern of the hard mask layer 113 may be removed by an oxidizing agent and an ammonium-based buffer, and the residue R may be removed by the etching controller, and at the same time, the metal layer 101 may be substantially not etched. After this, a bit line electrode or the like may be formed by filling the trench and via hole pattern of FIG. 5J with a metallic material, and/or the like.

### Examples 1 to 2, Comparative Examples 1 to 4 and 6

The materials listed in Table 1 as oxidizing agents, buffers, and etching controllers were weighed and mixed according to Table 1 to prepare compositions of Examples 1 and 2 and Comparative Examples 1 to 4 and 6. The remainder of each composition corresponds to water (deionized water). Examples 1 and 2 respectively included Compounds represented by 1 and 2 (as provided below) and Comparative Examples 1 to 4 and 6 respectively included Compounds represented by formulae B, D, E, C, and 2 (as provided below)

### Comparative Example 5

As an oxidizing agent and a buffer, the materials listed in Table 1 were weighed and mixed according to Table 1 to prepare a composition of Comparative Example 5. The remainder of each composition corresponds to water (deionized water).

### Evaluation Example 1

The composition of Example 1 was placed in each of three beakers and heated to 50° C., and then a copper layer, a cobalt layer, and a titanium nitride layer, which had been subjected to a plasma etching treatment, were immersed in the respective beakers for 10 minutes (copper layer), 5 minutes (cobalt layer) and 0.5 minutes (titanium nitride layer), and then the thickness of each of the copper layer and the cobalt layer was measured by using X-ray fluorescence spectrometry (XRF) (S8 Tiger, BRUKER), and the thickness of the titanium nitride layer was measured by using an ellipsometer (M-2000, JAWoolam). Accordingly, the etching rate with respect to the copper layer (hereinafter, “Cu layer etching rate”) (Å/min) of the composition of Example 1, the etching rate with respect to the cobalt layer (hereinafter, “Co layer etching rate”) (Å/min) of the composition of Example 1 and the etching rate with respect to the titanium nitride layer (hereinafter, “TiN layer etching rate”) (Å/min) of the composition of Example 1 were evaluated. Next, the TiN layer etching rate was divided by the Cu layer etching rate to evaluate R(TiN/Cu), and the TiN layer etching rate was divided by the Co layer etching rate to evaluate R(TiN/Co). Results thereof were summarized in Table 1.

This test was repeated using each of the compositions of Example 2 and Comparative Examples 1 to 6. Results are summarized in Table 1.

Next, a substrate having the trench and via hole pattern for forming a bit line electrode and having residue on the inner walls of the trench and via hole pattern was immersed for 5 minutes in a dip-type bath (25° C.) containing the composition of Example 1, and then a rinsing and drying process was performed. Then, whether the residue was removed was evaluated through atomic force microscopy (AFM) topography analysis. Results are summarized in Table 1. The substrate may be a substrate having a trench and via hole pattern formed as shown in FIG. 5I in which the metal layer 101 includes copper, the second dielectric layer 109 includes silicon oxide, the hard mask layer 113 includes titanium nitride, the buffer layer 111 includes carbon-doped silicon oxide, the second diffusion barrier layer 107 includes aluminum oxide, and the etching gas used in the dray etching process is CF₄.

The tests described above were repeated using each of the compositions of Example 2 and Comparative Examples 1 to 6. Results are summarized in Table 1.

TABLE 1

| | | | | | Cu | Co | TiN | | | |
|---|---|---|---|---|---|---|---|---|---|---|
| | | | | | layer | layer | layer | | | Whether |
| | Oxidizing | | Etching | | etching | etching | etching | R(TiN/ | R(TiN/ | to remove |
| Classification | agent | Buffer | controller | pH | rate | rate | rate | Cu) | Co) | residue |
| Example 1 | Hydrogen | (NH₄)₂HPO₄ | 1 | 7.5 | 0.2 | 0.3 | 165.2 | 826 | 550.7 | Acceptable |
| | peroxide | (0.5 wt %) | (0.2 | | | | | | | |
| | (25 wt %) | | wt %) | | | | | | | |
| Example 2 | Hydrogen | (NH₄)₂HPO₄ | 2 | 7.5 | 0.6 | 0.2 | 171.7 | 286.2 | 858.5 | Acceptable |
| | peroxide | (0.5 wt %) | (0.2 | | | | | | | |
| | (25 wt %) | | wt %) | | | | | | | |
| Comparative | Hydrogen | (NH₄)₂HPO₄ | B | 7.5 | 1.0 | 4.8 | 159.3 | 159.3 | 33.2 | Defective |
| Example 1 | peroxide | (0.5 wt %) | (0.2 | | | | | | | |
| | (25 wt %) | | wt %) | | | | | | | |
| Comparative | Hydrogen | (NH₄)₂HPO₄ | D | 7.5 | 0.6 | 1.7 | 149.2 | 248.7 | 87.8 | Defective |
| Example 2 | peroxide | (0.5 wt %) | (0.2 | | | | | | | |
| | (25 wt %) | | wt %) | | | | | | | |
| Comparative | Hydrogen | (NH₄)₂HPO₄ | E | 7.5 | 0.6 | 8.2 | 161.8 | 269.7 | 19.7 | Defective |
| Example 3 | peroxide | (0.5 wt %) | (0.2 | | | | | | | |
| | (25 wt %) | | wt %) | | | | | | | |
| Comparative | Hydrogen | (NH₄)₂HPO₄ | C | 7.5 | 2.4 | 4.1 | 158.4 | 66 | 38.6 | Defective |
| Example 4 | peroxide | (0.5 wt %) | (0.2 | | | | | | | |
| | (25 wt %) | | wt %) | | | | | | | |
| Comparative | Hydrogen | (NH₄)₂HPO₄ | — | 7.5 | 1.4 | >10 | 174.4 | 124.6 | 17.4 or | Defective |
| Example 5 | peroxide | (0.5 wt %) | | | | | | | less | |
| | (25 wt %) | | | | | | | | | |
| Comparative | Hydrogen | (NH₄)₂HPO₄ | 2 | 7.5 | 1.2 | 0.8 | 127.2 | 106 | 159 | Defective |
| Example 6 | peroxide | (0.5 wt %) | (0.2 | | | | | | | |
| | (15 wt %) | | wt %) | | | | | | | |

Acceptable: No residues with a length of 10 nm or more were observed.
Defective: Residues with a length of 10 nm or more were observed.

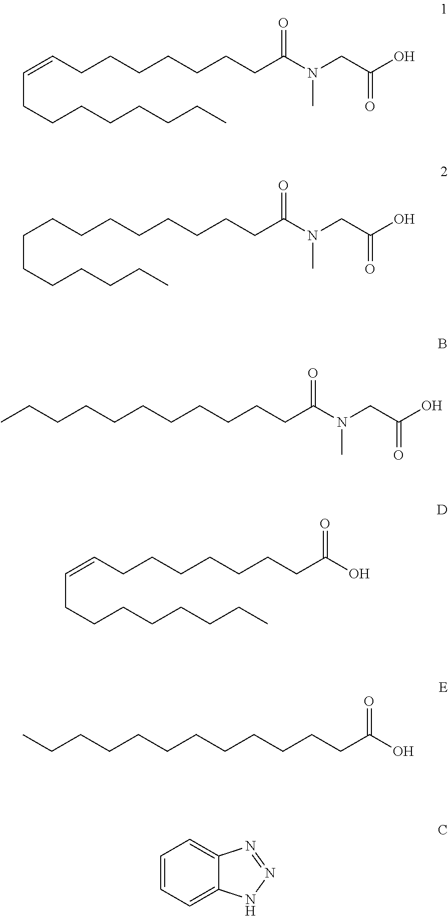 [SMILES: CCCCCCCC/C=C\CCCCCCCC(=O)N(C)CC(=O)O ; CCCCCCCCCCCCCCCC(=O)N(C)CC(=O)O ; CCCCCCCCCCCC(=O)N(C)CC(=O)O ; CCCCCCCC/C=C\CCCCCCCC(=O)O ; CCCCCCCCCCCCC(=O)O ; c1ccc2[nH]nnc2c1]

From Table 1, it can be seen that the compositions of Examples 1 and 2 may realize a high etching selectivity between a titanium nitride layer and a copper layer and a high etching selectivity between a titanium nitride layer and a cobalt layer, compared to the compositions of Comparative Examples 1 to 6, and at the same time, simultaneously exhibiting excellent performance in removing residues generated during a formation process and/or a patterning process of the metal-containing layer.

The compositions according to the disclosure have excellent etching selectivity and excellent cleaning performance, and thus may be effectively used in various processing processes for various metal-containing layers, such as etching and cleaning processes. Therefore, by processing a metal-containing layer using these compositions, a high-quality semiconductor device can be manufactured.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A composition comprising:
an oxidizing agent at about 16 wt % to about 50 wt % per 100 wt % of the composition;
an ammonium-based buffer; and
an etching controller, the etching controller comprising a compound represented by Formula 1: 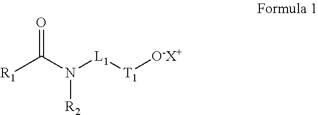
wherein, in Formula 1,
R₁ is a C₁₂-C₅₀ alkyl group or a C₁₂-C₅₀ alkenyl group,
R₂ is hydrogen, a C₁-C₅₀ alkyl group, or a C₂-C₅₀ alkenyl group,
L₁ is a C₁-C₂₀ alkylene group,
T₁ is a linker represented by one of 2(1) to 2(3), and
X is hydrogen, an alkali metal, or an ammonium group,
wherein at least one of methylene groups included in R₁ and R₂ is optionally substituted with O or S, and
wherein at least one of hydrogens included in R₁, R₂, and L₁ is optionally substituted with a halogen atom, a C₁-C₃₀ alkoxy group, or a C₁-C₃₀ alkylthio group, 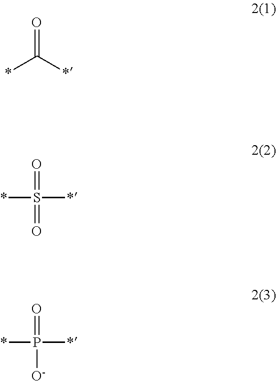
wherein, in Formulae 2(1) to 2(3),
* indicates a binding site to L₁, and
*′ indicates a binding site to O⁻.

2. The composition of claim 1, wherein
the oxidizing agent comprises hydrogen peroxide.

3. The composition of claim 1, wherein
an amount of the oxidizing agent is about 20 wt % to about 30 wt % of the composition.

4. The composition of claim 1, wherein
the ammonium-based buffer comprises a group represented by N(A₁₁)(A₁₂)(A₁₃)(A₁₄), and
A₁₁ to A₁₄ are each independently hydrogen, a C₁-C₃₀ alkyl group, a C₂-C₃₀ alkenyl group, a C₃-C₃₀ carbocyclic group, or a C₁-C₃₀ heterocyclic group.

5. The composition of claim 1, wherein the ammonium-based buffer does not comprise fluorine (F).

6. The composition of claim 1, wherein the ammonium-based buffer comprises at least one of hydroxide, acetate, bicarbonate, benzoate, carbonate, formate, nitrate, hydrogensulfate, carbamate, sulfamate, citrate, phosphate, sulfite, sulfobenzoate, oxalate, lactate, tartrate, dihydrogencitrate, glutamate, salicylate, bioxalate, octanoate, propionate, glycolate, or gluconate.

7. The composition of claim 1, wherein the ammonium-based buffer comprises a compound represented by Formula 11-1, a compound represented by Formula 11-2, a compound represented by Formula 11-3, a compound represented by Formula 11-4, or a combination thereof: 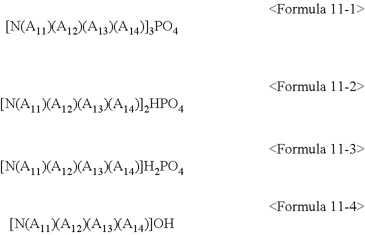
wherein, in Formulae 11-1 to 11-4, A₁₁ to A₁₄ are each independently hydrogen, a C₁-C₃₀ alkyl group, a C₂-C₃₀ alkenyl group, a C₃-C₃₀ carbocyclic group, or a C₁-C₃₀ heterocyclic group.

8. The composition of claim 1, wherein an amount of the ammonium-based buffer is about 0.01 wt % to about 10 wt % of the composition.

9. The composition of claim 1, wherein an amount of the ammonium-based buffer is about 0.01 wt % to about 4 wt % of the composition.

10. The composition of claim 1, wherein R₁ in Formula 1 is a C₁₃-C₂₀ alkyl group or a C₁₃-C₂₀ alkenyl group.

11. The composition of claim 1, wherein L₁ in Formula 1 is a C₁-C₄ alkylene group.

12. The composition of claim 1, wherein an amount of the etching controller is about 0.001 wt % to about 10 wt % of the composition.

13. A method of treating a metal-containing layer, the method comprising:
preparing a substrate, the substrate including the metal-containing layer; and
contacting the metal-containing layer with the composition of claim 1.

14. The method of claim 13, wherein
a metal included in the metal-containing layer comprises titanium (Ti), indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), or a combination thereof.

15. The method of claim 13, wherein
the metal-containing layer comprises a metal, a metal nitride, a metal oxide, a metal oxynitride, or a combination thereof, and
each of the metal, a metal of the metal nitride, a metal of the metal oxide, and a metal of the metal oxynitride comprises titanium (Ti), indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), or a combination thereof.

16. The method of claim 13, wherein the contacting of the metal-containing layer with the composition includes etching or cleaning at least a portion of the metal-containing layer.

17. The method of claim 13, wherein
the metal-containing layer comprises a first region and a second region, and
a second etching rate at which the composition etches the second region is greater than a first etching rate at which the composition etches the first region.

18. The method of claim 17, wherein
the first region comprises at least one of cobalt and copper, and
the second region comprises titanium nitride.

19. The method of claim 13, wherein
the contacting of the metal-containing layer with the composition includes removing residue on a surface of the metal-containing layer such that at least a portion of the metal-containing layer is cleaned, and
the residue comprises etching gas residue, polymer residue, metal-containing residue, or a combination thereof.

20. A method of manufacturing a semiconductor device, comprising:
preparing a substrate, the substrate including a metal-containing layer;
contacting the metal-containing layer with the composition of claim 1; and
performing a subsequent manufacturing process.
